(19) United States
(12) Patent Application Publication
(10) Pub. No.: US 2026/0034300 A1
(43) Pub. Date: Feb. 5, 2026

(54) BASAL RATE TESTING USING FREQUENT BLOOD GLUCOSE INPUT

(71) Applicant: Tandem Diabetes Care, Inc., San Diego, CA (US)

(72) Inventors: Michael L. Blomquist, Blaine, MN (US); Thomas Alan Savard, Arden, MN (US)

(21) Appl. No.: 19/351,391

(22) Filed: Oct. 7, 2025

Related U.S. Application Data
(63) Continuation of application No. 17/713,426, filed on Apr. 5, 2022, now Pat. No. 12,458,754.
Application No. 17/713,426 is a continuation of application No. 15/266,468, filed on Sep. 15, 2016, now Pat. No. 11,291,763.
Application No. 15/266,468 is a continuation of application No. 11/685,617, filed on Mar. 13, 2007, now abandoned.

(51) Int. Cl. A61M 5/172; A61B 5/00; A61B 5/145; A61M 5/142; G16H 20/10; G16H 40/63

(52) U.S. Cl. CPC A61M 5/1723; A61M 5/14244; G16H 20/10; A61B 5/14503; A61B 5/14532; A61B 5/4839; A61M 2005/14208; A61M 2205/502; A61M 2230/005; A61M 2230/201; G16H 40/63

(57) ABSTRACT

An apparatus comprising a user interface configured to generate an electrical signal to start a basal insulin rate test when prompted by a user, an input configured to receive sampled blood glucose data of a patient that is obtained during a specified time duration, including a time duration during delivery of insulin according to a specified basal insulin rate pattern, and a controller communicatively coupled to the input and the user interface. The controller includes an insulin calculation module configured for determining at least one of an amount of basal insulin over-delivered and an amount of basal insulin under-delivered during the basal insulin rate test in trying to meet a target blood glucose baseline. Other devices and methods are disclosed.

### RELATED APPLICATION

This application is a continuation of U.S. application Ser. No. 17/713,426 filed Apr. 5, 2022, which is a continuation of U.S. application Ser. No. 15/266,468 filed Sep. 15, 2016, which is a continuation of U.S. application Ser. No. 11/685,617 filed Mar. 13, 2007, each of which are hereby fully incorporated herein by reference.

### TECHNICAL FIELD

The field generally relates to patient insulin management devices and, in particular, but not by way of limitation, to systems, devices and methods for adjusting insulin therapy.

### BACKGROUND

People who suffer from diabetes require insulin to keep their blood glucose level as close as possible to normal levels. It is essential for people with diabetes to manage their blood glucose level to within a normal range. Complications from diabetes can include heart disease (cardiovascular disease), blindness (retinopathy), nerve damage (neuropathy), and kidney damage (nephropathy). Insulin is a hormone that reduces the level of blood glucose in the body. Normally, insulin is produced by beta cells in the pancreas. In non-diabetic people, the beta cells release insulin to satisfy two types of insulin needs. The first type is a low-level of background insulin that is released throughout the day. The second type is a quick release of a higher-level of insulin in response to eating. Insulin therapy replaces or supplements insulin produced by the pancreas.

Conventional insulin therapy typically involves one or two injections a day. The low number of injections has the disadvantage of allowing larger variations in a person's insulin levels. Some people with diabetes manage their blood glucose level with multiple daily injections (MDI). MDI may involve more than three injections a day and four or more blood glucose tests a day. MDI offers better control than conventional therapy. However, insulin injections are inconvenient and require a diabetic person to track the insulin doses, the amount of carbohydrates eaten, and their blood glucose levels among other information critical to control.

Blood glucose (BG) management devices help a diabetic person manage their blood glucose. For example, an insulin pump is a BG management device that provides insulin throughout the day. A glucose monitor (GM) or meter is a BG management device that measures blood glucose levels. Some GMs require a finger-stick to acquire a sample of blood that is applied to a test strip to get a blood glucose reading. Some GMs are able to provide continuous monitoring of blood glucose. Other BG management devices include computers running software to help a diabetic person manage insulin therapy. However, most BG management devices are limited in the control over blood glucose that they offer.

### SUMMARY

This document discusses, among other things, devices and methods for managing insulin therapy. A device example includes a user interface configured to generate an electrical signal to start a basal insulin rate test when prompted by a user, an input configured to receive sampled blood glucose data of a patient that is obtained during a specified time duration, including a time duration during delivery of insulin according to a specified basal insulin rate pattern, and a controller communicatively coupled to the input and the user interface. The controller includes an insulin calculation module configured for determining at least one of an amount of basal insulin over-delivered and an amount of basal insulin under-delivered during the basal insulin rate test in trying to meet a target blood glucose baseline.

A method example includes receiving a user prompt in a blood glucose (BG) management device to start a basal insulin rate test, receiving sampled blood glucose data that is obtained during a specified duration of time when insulin is delivered according to a specified basal insulin rate pattern, and determining at least one of an amount of basal insulin over-delivered and an amount of basal insulin under-delivered in trying to meet a target blood glucose baseline during the basal insulin rate test using the BG management device.

This summary is intended to provide an overview of the subject matter of the present patent application. It is not intended to provide an exclusive or exhaustive explanation of the invention. The detailed description is included to provide further information about the subject matter of the present patent application.

### BRIEF DESCRIPTION OF THE DRAWINGS

FIG. 1 is a block diagram of portions of a BG management device.

FIG. 2 shows example illustrations of a blood glucose concentration graph and a basal rate pattern.

FIG. 3 is a block diagram of portions of an example of a BG management device that includes a pump mechanism.

FIG. 4 is an illustration of a BG management device that includes an insulin pump.

FIG. 5 is another block diagram of portions of a BG management device that includes a pump mechanism.

FIG. 6 is a block diagram of a BG management device that includes a blood glucose sensor circuit.

FIG. 7 is a block diagram of portions of another example of a BG management device.

FIG. 8 is a flow diagram of a method of using a BG management device to execute a basal rate test.

FIG. 9 is a flow diagram of another method of using a BG management device to execute a basal rate test.

### DETAILED DESCRIPTION

In the following detailed description, reference is made to the accompanying drawings which form a part hereof, and specific embodiments in which the invention may be practiced are shown by way of illustration. It is to be understood that other embodiments may be used and structural or logical changes may be made without departing from the scope of the present invention.

It is important for a diabetic person to be treated with the proper amount of insulin. As discussed previously, high blood sugar can lead to serious complications. Conversely, a person with low blood sugar can develop hypoglycemia. Ideally, insulin therapy mimics the way the body works. An insulin pump is one way to mimic the body's insulin production. An insulin pump can provide a background or basal infusion of insulin throughout the day and provide a quick release or bolus of insulin when carbohydrates are eaten. If a person develops high blood sugar, a correction bolus can be delivered by the pump to correct it. While insulin pumps improve convenience and flexibility for a diabetic person, they can be sophisticated devices. Some insulin pumps can be difficult to program. Proper use of an insulin pump requires a user to go through a learning curve to properly use and program the pump.

Basal rate refers to a type of twenty-four hour background infusion of insulin by an insulin pump that mimics the continuous background release of insulin from a normal pancreas. It is the rate of insulin delivery the patient normally needs independent of the consumption of meals. The basal rate is typically specified in insulin units per hour (u/hr). Typically, a basal rate for a pump is initially programmed by a clinician based on a total daily dose (TDD) of insulin for a diabetic person. The clinician may determine TDD based on many factors including the type of diabetes of the patient and the patient's weight, age, and level of fitness. The amount of basal insulin is typically determined to be a percentage of TDD, such as 40%, 50%, or 60% for example. The total daily dose is then divided by 24 to obtain an average basal rate. For example, if a patient's TDD is determined to be 40 units of insulin, and 50% of the TDD is used for basal delivery, the average basal rate is 20 units/24 hours or 0.83 u/hr.

Many insulin pump users may use three or more different basal rates during the course of a day. Basal rates can be adjusted to change delivery every few minutes (e.g., 20-30 minutes) by increments as small as 0.05 u/hr to better track changes in demand, such as from an increase typically needed before dawn or a decrease needed during long active periods. Insulin pump users may use different basal rates for overnight, for breakfast to mid-afternoon, and for mid-afternoon to bedtime. Appropriate basal rates vary from person to person, may be different for a person at various times of the day, and may change for a person over time. Inappropriate basal rate settings may result in low blood glucose levels overnight or high blood glucose levels in the morning. An insulin pump user may go through several iterations of trial and error before finding appropriate basal rates. Because a patient's basal insulin needs may change over time, such as with weight change or with a change in fitness level, basal rate testing may be performed periodically to ensure that an appropriate basal rate is being delivered by an insulin pump. Blood glucose (BG) management devices are more valuable to a diabetic person if the device conveniently assists them in determining their appropriate basal rate or rates.

### Apparatus Embodiments

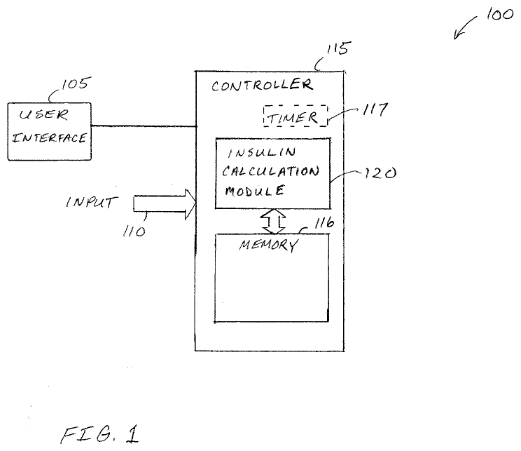

FIG. 1 is a block diagram of portions of a BG management device 100. Examples of a BG management device 100 include, among other devices, an insulin pump, a blood glucose monitor (GM) or meter, and a computing device running software to assist a diabetic patient in managing insulin therapy. Examples of a computing device include, among other things, a personal computer or a personal data assistant (PDA).

The BG management device 100 includes a user interface 105, an input 110, and a controller 115 communicatively coupled to the input 110 and the user interface 105. The controller 115 can be implemented using hardware circuits, firmware, software or any combination of hardware, firmware and software. Examples, include a microcontroller, a logical state machine, and a processor such as a microprocessor, application specific integrated circuit (ASIC), or other type of processor.

The user interface 105 generates an electrical signal to begin a basal rate test when prompted by a user. The user interface 105 may include a pushbutton, keypad, or a computer mouse. The user interface 105 may include a display operatively coupled to the controller 115 to provide patient or user instructions for the basal rate test. Examples of instructions include, among other things, instructing the patient not to eat during the test, to maintain a normal activity level, and not to administer an insulin correction bolus during the test. The display may include a touch-screen. The user of the device may be a clinician, caregiver, or a diabetic patient. The user prompts the BG management device 100 using the user interface 105 to begin a basal rate test. The basal rate test assists the user in determining one or more appropriate basal rates.

As part of a basal rate test, the patient receives insulin according to a specified basal rate pattern or profile. If the BG management device 100 includes an insulin pump, the basal insulin may be delivered using the BG management device 100. If the BG management device 100 does not include an insulin pump, the basal insulin may be delivered using a separate device that includes an insulin pump.

If the BG management device 100 includes an insulin pump, the BG management device 100 may further include a memory 116 to store at least one basal rate pattern. The controller 115 may display instructions for the user to enter one or more basal rates to be delivered according to time of day. For example, the BG management device 100 may allow the user to enter basal rate values in 0.05 u/hr increments, and to enter time in increments of one-half hour throughout the day. In some embodiments, the BG management device 100 stores different basal rate patterns according to different segments of the day, such as early in the day, late in the day, and overnight for example. In some embodiments, the input 110 may include a communication port and a basal rate pattern may be loaded from a second device into memory 116.

The input 110 is configured to receive sampled blood glucose data of the patient as part of the basal rate test. The blood glucose data provides an indication of the concentration level of the patient's blood sugar and the data may be obtained from blood directly or from insterstitial fluid. The blood glucose data is obtained during a specified time duration. The specified time duration includes a time when insulin is delivered according to a specified basal rate pattern, but may include a time prior or after the delivery of insulin as well. The configuration of the input 110 may depend on the type of BG management device 100. If the BG management device 100 is an insulin pump, the input 110 may be coupled to a GM included in the pump or the input 110 may include a communication port to receive the blood glucose data from a second device. The second device may include a GM or the second device may receive the blood glucose data from a third device. In some embodiments, the input 110 is coupled to the user interface 105, and the user may manually input the data into the pump through a keypad or keyboard included in the user interface.

The controller 115 includes an insulin calculation module 120. Modules can be software, hardware, firmware or any combination of software, hardware, and firmware. Multiple functions can be performed in one or more modules. The insulin calculation module 120 determines at least one of an amount of basal insulin over-delivered and an amount of basal insulin under-delivered during the basal insulin rate test in trying to meet a target blood glucose baseline.

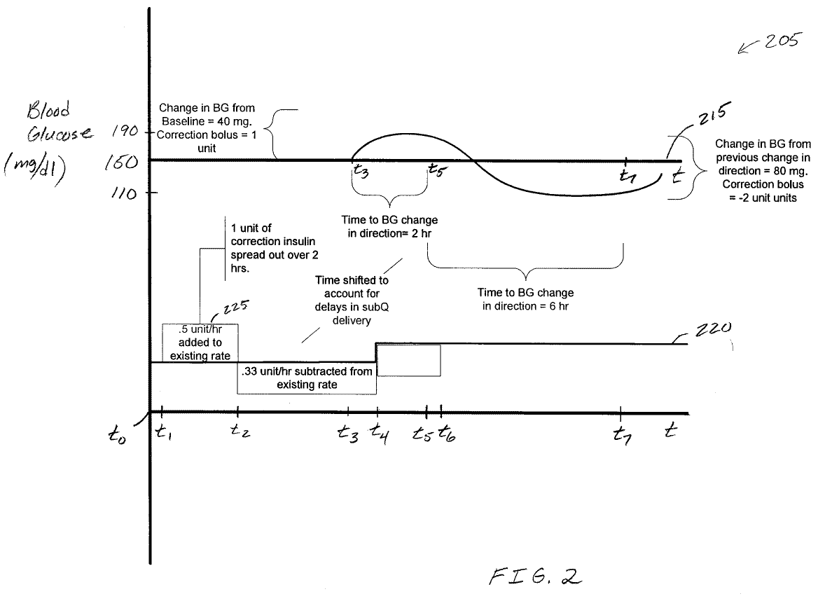

FIG. 2 shows example illustrations (not real data) of a blood glucose concentration graph 205 and a basal rate pattern 220 or profile during a basal rate test. Assume, as shown in the blood glucose concentration graph 205, that the patient's target blood glucose baseline 215 is 150 mg/dl (milligrams per deciliter) and that this is the patient's blood glucose concentration level before the basal rate test. Basal insulin is being delivered according to a basal rate pattern 220. At time to, the user elects to begin a basal rate test. User instructions for the basal rate test may be provided. The blood glucose concentration is determined from blood glucose data received into the input 110 during the basal rate test. The basal rate test may run over several hours, e.g., six to eight hours. In some embodiments, the blood glucose data may be stored in memory for processing. In some embodiments, the blood glucose data may be processed by the insulin calculation module 120 as it is received.

If the patient's blood glucose level remains at the target blood glucose baseline 215 or within a specified range of the target blood glucose baseline 215, the basal profile is appropriate. If the patient's blood glucose level rises above the target blood glucose baseline 215 or rises above a specified range of the target blood glucose baseline 215, the basal rate is too low and there was an under-delivery of basal insulin. If the patient's blood glucose level falls below the target blood glucose baseline 215 or falls below a specified range of the target blood glucose baseline 215, the basal rate is too high and there was an over-delivery of basal insulin.

In the example in FIG. 2, the patient's blood glucose begins to rise at time t₃. The change in the blood glucose from the baseline reaches 190 mg/dl, or an increase of 40 mg/dl. At time t₄, the basal rate pattern 220 includes an increase in basal rate. The blood glucose level of the patient begins to change direction, here a decrease, at time t₅. The time duration from the increase at t₃ to the change in direction at t₅ is about two hours in this example. The blood glucose level of the patient eventually falls to 110 mg/dl, or a total decrease of 80 mg/dl. At time t₇, the blood glucose level of the patient begins to again change direction. This time the change in direction is an increase in blood glucose concentration. The time duration from the decrease at t₅ to the change in direction at t₇ is about six hours.

In some embodiments, the insulin calculation module 120 is configured to determine the over-delivered amount or the under-delivered amount of basal insulin using a correction factor of the patient and a variance of a blood glucose level from the target blood glucose baseline 215. A correction factor refers to the amount of drop in blood glucose concentration of the patient for one unit of insulin. In FIG. 2, the 40 mg/dl increase corresponds to an under-delivery of basal insulin. The under-delivery may be due to the basal rate being too low or due to an increased demand from the patient during that time of day. The 80 mg/dl decrease corresponds to an over-delivery of basal insulin.

To calculate the amount under-delivered, the insulin calculation module 120 divides the increase in blood glucose level (+40 mg/dl) by the correction factor of the patient to determine the amount of insulin required to lower the blood glucose level to the target blood glucose baseline 215. This is the amount of insulin that was under-delivered to the patient during the basal rate test. Assume in the example of FIG. 2 that the patient's correction factor is one unit per 40 mg/dl. In this case, a correction bolus of one unit of insulin would decrease the patient's blood glucose level to the blood glucose baseline. To calculate the amount of insulin over-delivered, the insulin calculation module 120 divides the decrease in blood glucose level (−80 mg/dl) by the correction factor of the patient (1 u per 40 mg/dl). This corresponds to a correction bolus of −2 units of insulin, i.e., the amount of insulin delivered needs to be reduced by 2 units of insulin.

The under-delivered or over-delivered amount can be used to recommend changes to the basal rate pattern. In the example of FIG. 2, the insulin calculation module 120 may determine that the existing basal rate pattern 220 needs to be increased at some point by one unit of insulin to address the 40 mg/dl increase and decreased at some point by 2 units of insulin to address the 80 mg/dl decrease.

The BG management device 100 is more valuable if recommended changes anticipate an under-delivery or over-delivery. However, anticipating when to change the basal rate is complicated by a delay, or a lag time, in insulin uptake before the insulin becomes effective. Another complication is that the lag time may be different for glucose levels measured using blood and glucose levels measured using interstitial fluid. Measuring blood glucose concentration using the interstitial fluid may make the uptake appear to have additional lag time. In some embodiments, the insulin calculation module 120 recommends a change in a basal rate that precedes any actual times of under-delivery or over-delivery by a time duration that compensates for a lag time associated with the subcutaneous insulin delivery and with the glucose measurement method.

In some embodiments, in addition to the uptake lag time, the insulin calculation module 120 uses the time from a beginning of a change in the blood glucose level to a change in direction of the blood glucose data values to determine a recommended change to the basal insulin rate pattern 220. In the example of FIG. 2, it is determined that one unit of insulin is needed to correct the under-delivery of insulin resulting in the 40 mg/dl increase in blood glucose level. The increase began at t₃ and a change in direction occurred two hours later at t₅. The insulin calculation module 120 may recommend a change that includes adding one unit of insulin to the basal rate pattern 220 and spreading the delivery out over two hours corresponding to the change in direction time, i.e., a rate of 0.5 u/hr. This shown by the basal rate increase 225 of 0.5 u/hr for two hours over time t₁ to t₂. The time t₁ is shifted earlier than the time of the increase at t₃ by a time duration to compensate for a delay in the insulin uptake so that the insulin may act on the blood glucose.

Also in FIG. 2, an over-delivery of 2 units of insulin resulted in an 80 mg/dl increase in blood glucose level. The decrease began at t₅ and a change in direction occurred six hours later at t₇. The insulin calculation module 120 may recommend a change that includes subtracting two units of insulin from the basal rate pattern 220 over six hours at a rate of 0.33 u/hr. This shown by the basal rate decrease 230 of 0.33 u/hr for six hours over time t₂ to t₆. The time t₂ is early enough to compensate for the delay in insulin uptake.

The lag time for insulin uptake may depend on several factors. In some embodiments, the insulin calculation module 120 determines a time duration to compensate for such a time lag using the type of insulin delivered. Some insulin types have a faster uptake than other types, and the insulin calculation module 120 may use a table stored in a memory of the BG management device to correlate a time duration to an insulin type. In some embodiments, the insulin calculation module 120 calculates the compensating time duration using an activity level of the patient and/or the fitness level of the patient. In some embodiments, the compensating time lag is pre-determined from clinical studies and is stored in a memory for use by the insulin calculation module 120.

In some embodiments, the insulin calculation module 120 may adjust the correction factor before determining an amount of insulin under or over-delivered. In certain embodiments, the insulin calculation module 120 may use a correction factor multiplier to adjust the correction factor when determining the amount of insulin under or over-delivered, and consequently adjusting the amount of insulin in any recommended changes to the basal rate pattern 220. For example, assume as in FIG. 2 that the patient's correction factor is one unit per 40 mg/dl. If the correction factor multiplier is 1.3, the insulin calculation module uses a correction factor of one unit per 52 mg/dl [(1.3)(40 mg/dl/unit)]. For the 40 mg/dl increase in FIG. 2, the insulin calculation module 120 divides the increase in blood glucose level (40 mg/dl) by the correction factor of the patient (1 u per 52 mg/dl). This corresponds to a correction bolus of 0.77 units [(40)/(52)] of insulin. The insulin calculation module 120 may recommend adding 0.39 u/hr for two hours to the basal rate pattern.

Using a correction factor multiplier results in a lower amount of basal insulin allowing adjustments to be made more safely made. This may give a user more confidence in using the recommended changes to the basal rate pattern 220. The 80 mg/dl decrease corresponds to a correction bolus of 1.54 units of insulin. The insulin calculation module 120 may recommend subtracting 0.26 u/hr for six hours to the basal rate pattern. The controller 115 may store the correction factor multiplier in a memory. The correction factor multiplier may be manually set or programmed by a clinician. The clinician may set the correction factor multiplier to a value that accords to a level of confidence or comfort to the clinician in the recommended changes to the basal rate pattern 220.

In some embodiments, if the blood glucose data received during the basal rate test indicates that the blood glucose level of the patient is outside of a specified range of blood glucose levels, the controller 115 cancels the basal insulin rate test. If the blood glucose level is above the range, the controller 115 may recommend a correction bolus to be taken by the patient. The insulin calculation module 120 calculates the amount of insulin in the correction bolus by dividing the blood glucose concentration by the specified correction factor for the patient.

If the blood glucose level is below the range, the controller 115 may recommend an amount of carbohydrates to be eaten by the patient. The insulin calculation module 120 calculates the amount of carbohydrates using a correction factor specified for the patient and a carbohydrate ratio specified for the patient. A carbohydrate ratio refers to the amount of carbohydrates reduced, or covered, by a unit of insulin.

For example, assume that at the beginning of a basal rate test, the blood glucose level of a patient is 40 mg/dl below the specified range and the specified correction factor is 1 unit per 80 mg/dl. The insulin calculation module 120 determines that −0.5 units of insulin (−40/80) are required to bring the blood glucose level back within the specified range. Negative insulin cannot be delivered so this corresponds to a requirement for carbohydrates. Assume that the carbohydrate ratio of the patient is 20 grams of carbohydrates per unit of insulin (20 g/u). The insulin calculation module 120 multiplies the amount of insulin by the carbohydrate ratio to determine that the patient should eat 10 grams of carbohydrates [(0.5)(20)]. The insulin calculation module 120 may take into account additional factors such as the health status of the patient and the activity level of the patient in recommending the carbohydrate amount. In some embodiments, if the blood glucose of the patient is outside the specified range of blood glucose levels, the controller 115 suspends the start of the basal insulin rate test until the blood glucose of the patient is within the specified range of blood glucose levels.

As discussed previously, appropriate basal rates may differ for a patient throughout the course of a day. The BG management device 100 may include a timer circuit 117 operatively coupled to the controller 115. The controller 115 displays user instructions to execute a basal rate test at one or more specified times during a day. In some embodiments, controller 115 displays user instructions to run the basal insulin rate test on multiple days. The controller 115 may prompt the user to run the test during substantially the same time on the multiple days. This may result in more appropriate basal delivery rates being used at different times during the day.

It is often difficult to maintain a stable blood glucose target value overnight because the correction factor varies as a function of time. In order to stabilize the glucose value at a target blood glucose value, the basal rate may often be adjusted during overnight periods to compensate for changes in the correction factor. An insulin pump user may go through several iterations of trial and error while attempting to find appropriate overnight basal rates. A trial and error method may result in less than optimal control of overnight blood glucose level.

According to some embodiments, the BG management device 100 automatically executes a basal rate test during a period when food intake is restricted, such as overnight for example. The basal rate test may start a specified time after a user prompts the BG management device 100 to execute the basal rate test. For example, if the period is overnight, the user prompt may start a timer circuit and the controller 115 may initiate the overnight basal rate test when a time duration expires. The insulin calculation module 120 automatically determines one or more basal rates for a basal rate profile using a basal rate calibration and verification technique. The basal blood glucose value g can be approximated by

$$g ( t ) ≅ c ( t ) b ( t - τ ) , \qquad ( 1 )$$

where c(t) is the basal correction factor, b(t) is the basal insulin rate, and r is the delay or lag time associated with the uptake of a subcutaneous infusion of insulin. Food consumption and exercise are assumed to be negligible during the period of the test.

The insulin calculation module 120 may perform a rapid calibration that can be executed during a period as short as two time periods, such as two nights for example. The correction factor c(t) may vary as a function of time. To determine c(t), blood glucose data values g₁(t) and basal insulin delivery rates b₁(t) are recorded periodically throughout a first observation period. Rewriting Equation (1) to solve for c(t) for the first period yields

$${c}_{1} ( t ) = \frac{{g}_{1} ( t )}{{b}_{1} ( t + {τ}_{1} )} . \qquad ( 3 )$$

The delay for insulin uptake τ₁ can be an assumed value based on current estimates from clinical studies that use that type of insulin, or can be determined on a per patient basis using stochastic or deterministic time series analysis of prior or current basal test data. The time series analysis of the blood glucose data values may be performed under pulse function, step function, or continuous changes in insulin delivery. The time-dependent changes in insulin delivery may be present in the user's current basal profile or the user may be prompted to create a time-dependent change by the insulin calculation module. The stochastic or deterministic time series analysis can be performed on blood glucose data obtained from previous calibration or observation periods, such as previous nights for example. Thus, the delay for insulin uptake may be determined using blood glucose data obtained prior to the basal rate test.

A desired target blood glucose value gₜ(t) may be a constant or a function of time. Equation (1) can be written as

$$g ( t ) ≅ {c}_{1} ( t ) {b}_{t} ( t - {τ}_{1} ) , \qquad ( 3 )$$

where cₗ(t) is the correction factor determined from the first period of data values from Equation (2). Solving equation (3) for a controlling basal insulin rate bₜ(t) that achieves the desired gₜ(t) yields

$${b}_{t} ( t ) = \frac{{g}_{t} ( t + {τ}_{1} )}{{c}_{1} ( t + {τ}_{1} )} . \qquad ( 4 )$$

It is assumed that the correction factor c(t) is a periodic function that repeats on a twenty four hour cycle, and that c(t) determined from data and basal rates during the first period of reduced food intake will be similar on subsequent periods twenty-four hours later.

During the second period of observation, blood glucose data values g₂(t) and basal rates b₂(t) are again recorded periodically. Ideally g₂(t)=gₜ(t), but in reality g₂(t)=gₜ(t)+ε(t), where ∈(t) is the residual deviation from the target blood glucose value. Thus, Equation (1) can be written as

$${g}_{t} ( t ) + ε ( t ) = {c}_{1} ( t ) {b}_{1} ( t - {τ}_{1} ) . \qquad ( 5 )$$

Assuming that ε(t) is primarily due to the error in the estimate of τ₁, Equation (5) can be rewritten as

$${g}_{t} ( t ) + ε ( t ) = {c}_{1} ( t ) {b}_{t 1} ( t - ( {τ}_{1} - δ ) ) , \qquad ( 6 )$$

where δ is the error in the delay estimate. Combining Equations 5 and 6 gives

$$ε ( t ) = {c}_{1} ( t ) [ {b}_{t} ( t - {τ}_{1} ) - {b}_{t} ( t - ( {τ}_{1} - δ ) ) ] . \qquad ( 7 )$$

Curve fitting or other standard minimization techniques can be used to determine the most appropriate estimate of δ to satisfy Equation (7). Once δ is determined, the control estimate for the basal insulin delivery rate or rates bₜ(t) that achieves the desired blood glucose target gₜ(t) can be written as

$${b}_{t} ( t ) = \frac{{g}_{t} ( t + {τ}_{2} )}{{c}_{1} ( t + {τ}_{2} )} , \qquad ( 8 )$$

where τ₂=τ₁−δ. The insulin calculation module may then recommend changes to the t basal rate pattern using bₜ(t).

The rapid calibration technique is a method to quickly achieve improved control over blood glucose level. In some embodiments, the insulin calculation module 120 executes a basal rate test that uses a generalized calibration technique to achieve more accurate estimates of bₜ(t). The generalized calibration method uses least squares estimation techniques with at least two periods of observing blood glucose data and basal insulin delivery rates. Referring back to Equation (1) and with g(t) and b(t) measured over several periods, r and c(t) can be estimated by curve fitting with a finite order polynomial or an orthogonal series approximation such as a Fourier series approximation for example. The resulting estimate of bₜ(t) is calculated using Equation 3 with r and c(t) estimated from the curve fit results.

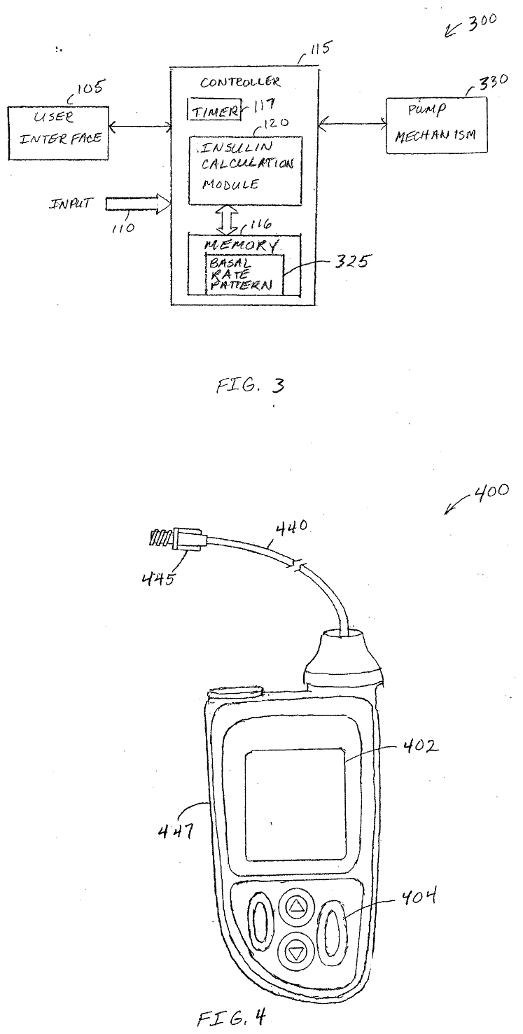

According to some embodiments, the BG management device includes an insulin pump. FIG. 3 is a block diagram of portions of an example of a BG management device 300 that includes a pump mechanism 330 to deliver insulin to the patient. The pump mechanism 330 is operatively coupled to the controller 115. The controller 115 may track the amount of insulin delivered via the pump mechanism 330. The BG management device 300 includes a memory 116 operatively coupled to the controller 115 to store one or more basal rate patterns 325. The BG management device delivers basal insulin according to the basal rate patterns. The BG management device 300 also may deliver insulin through boluses such as a correction bolus or a carbohydrate bolus. In some embodiments, the BG management device 300 has a timer circuit 117 that includes a real time clock coupled to the controller 115. The controller 115 is configured to vary a basal rate of insulin delivery by a time of day according to a basal rate pattern.

In some embodiments, the insulin calculation module 120 is able to keep track of the amount of active insulin in the patient. This is sometimes referred to as insulin on board (IOB). To track the amount of active insulin, the controller 115 uses the amount of insulin delivered, the time that elapsed since delivery of insulin and a duration of how long the insulin is active in the blood. The duration may be determined using kinetic action, which is the time it takes for insulin to disappear from the blood, or the duration of insulin action (DIA), which is how long the insulin lowers blood glucose. In some embodiments, the controller 115 cancels a basal rate test if the insulin calculation module 120 determines that the active insulin amount is above a specified threshold insulin amount. This minimizes the risk of IOB confounding the results of the basal rate test.

In some embodiments, the controller 115 cancels the basal insulin rate test if the controller 115 determines that an insulin bolus dose, such as a correction insulin bolus or a carbohydrate insulin bolus, is delivered during the basal insulin rate test. In some embodiments, if the user enables an insulin bolus delivery, the controller 115 displays a warning that the basal insulin test will be canceled if the user elects to proceed with delivery of the insulin bolus dose.

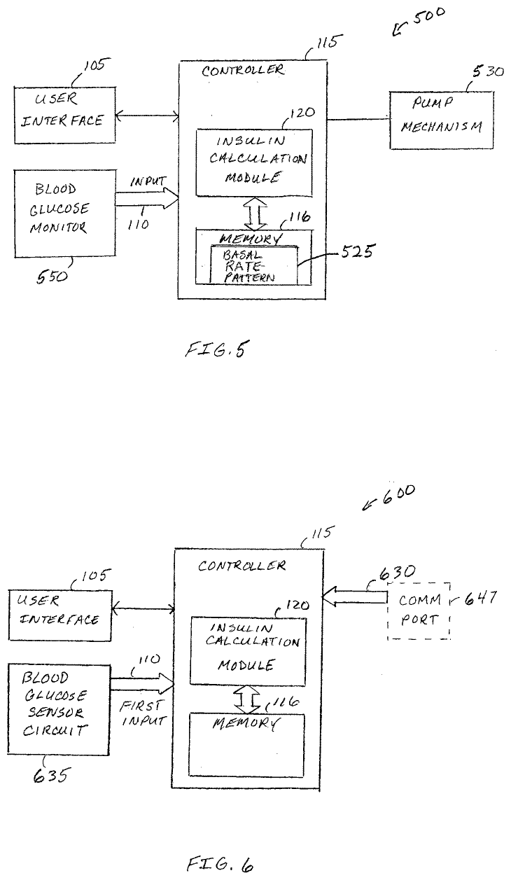

FIG. 4 is an illustration of a BG management device 400 that includes an insulin pump. The BG management device 400 includes a cassette or cartridge of insulin and tubing 440 connectable to a patient such as by a Luer lock 445. The BG management device 400 includes a user interface that may include a display 402 operatively coupled to a controller 115. The user interface may also include one or more keys 404.

Returning to FIG. 3, the blood glucose data obtained during the basal insulin rate test may be produced by a second device separate from the BG management device 300. The controller 115 displays user instructions for the basal rate test. The user interface 105 and the input 110 are configured to receive the sampled blood glucose data entered manually by the user through the user interface 105. The controller 115 may periodically prompt the user to enter a blood glucose value at different times during the test, or to enter the blood glucose data all at once after the test.

FIG. 5 is another block diagram of portions of a BG management device 500 that includes a pump mechanism 530 and delivers basal insulin according to one or more basal rate patterns 525 stored in memory 116. A blood glucose monitor, or GM 550, is communicatively coupled to the input 110. The input 110 is configured to receive the sampled blood glucose data from the GM 550. In some examples, the GM 550 is included in the BG management device 500 and is coupled to the input 110. In some examples, the GM 550 is included in a second device. The input 110 may receive the blood glucose data during the basal rate test or after the test is run. The input 110 may include a communication port, such as communication port 447 located on the rear face of the device in FIG. 4, and the GM 550 is communicatively coupled to the input 110 by the communication port 447. In some embodiments, the communication port 447 is a wired port such as a serial interface or bus interface for communicating with the second device. In some embodiments, the communication port 447 is a wireless port such as an infrared (IR) communication port or a radio frequency (RF) communication port. The input 110 wirelessly receives the sampled blood glucose data from the second device.

Returning to FIG. 5, in some embodiments, the GM 550 is a continuous GM and automatically collects the sampled blood glucose data. For example, the GM 550 may include a blood glucose sensor. The blood glucose sensor produces a blood glucose signal representative of a blood glucose level of the patient. The GM 550 samples the blood glucose signal to obtain the sampled blood glucose data.

In some embodiments, the GM 550 may need to prompt the user to begin a blood glucose measurement. For example, the GM 550 may require diabetes test strips to take a blood glucose measurement. The controller 115 prompts the user, via a display, to begin a blood glucose measurement using the GM 550. The user then provides a new test strip to the GM 550 when prompted during the basal rate test. In another example, the GM 550 may include a drum of diabetes test strips and the user advances the drum to a fresh or unused test strip when prompted by the controller 115. The controller 115 may display a recommended basal rate after the basal rate test. The controller 115 may also communicate a recommended change in the basal rate to the second device via a communication port.

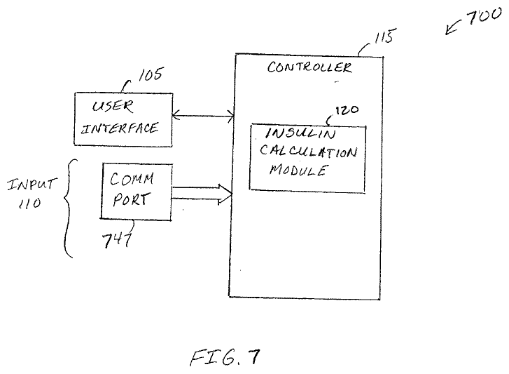

According to some embodiments, the BG management device is a GM. FIG. 6 is a block diagram of a BG management device 600 that includes a blood glucose sensor circuit 635 operatively coupled to the input 110. The blood glucose sensor circuit 635 produces a blood glucose signal representative of a blood glucose level of the patient and provides the sampled blood glucose data to input 110. In some embodiments, the blood glucose sensor circuit 635 includes an implantable blood glucose sensor. In some embodiments, the blood glucose sensor includes a percutaneous blood glucose sensor. The blood glucose sensor circuit 635 may include signal conditioning circuits, such as for signal filtering and signal amplification for example. If an implantable blood glucose sensor is used, the blood glucose sensor circuit 635 may include a communication circuit configured to receive blood glucose data wirelessly, such as by RF communication.

The BG management device 600 includes a second input 630 communicatively coupled to the controller 115. The second input 630 receives information related to basal insulin delivery, such as one or more basal rate patterns used during the basal rate test. The information related to insulin delivery may be received into a memory 116. The insulin calculation module 120 determines at least one of an amount of insulin over-delivered and an amount of insulin under-delivered during the basal rate test using the insulin delivery information and the sampled blood glucose data. The BG management device 600 may include a communication port 647 coupled to the second input 630. The communication port 647 receives the information related to insulin delivery from a second device. In some embodiments, the communication port 647 is a wired port such a serial interface or bus interface. In some embodiments, the communication port 647 is a wireless port such as an infrared (IR) communication port or a radio frequency (RF) communication port. The second input 630 wirelessly receives the insulin delivery data from the second device. As an example, the second device may be an insulin pump. The insulin calculation module 120 may determine changes to the basal rate pattern used to deliver basal insulin during the basal rate test. The controller 115 communicates recommended changes through the communication port 647 or may display the recommended changes on a display.

In some embodiments, the user interface 105 and the second input 630 are configured to receive the information related to insulin delivery by a user manually entering the information through the user interface 105. The insulin delivery information may be obtained from a pump for example. The controller 115 may display any recommended changes to the basal rate pattern.

FIG. 7 is a block diagram of portions of another example of a BG management device 700. BG management device 700 includes neither a GM nor an insulin pump. The BG management device 700 includes a user interface 105, an input 110, and a controller 115 communicatively coupled to the input 110 and the user interface 105. The input 110 includes at least one communication port 747 configured for receiving sampled blood glucose information. The communication port 747 may provide a wired connection to a second device, or the communication port 747 may provide a wireless connection to a second device. The sampled blood glucose information may include at least one time-stamp in order to align the sampled blood glucose information to information related to insulin delivery.

The insulin delivery information may be received through the same communication port 747 or a second communication port. The communication ports may be any combination of wired or wireless communication ports. The insulin delivery information includes information related to basal insulin delivered according to a basal rate pattern, and may include at least one time-stamp to align the insulin delivery information with the blood glucose information. The insulin calculation module 120 determines at least one of an amount of insulin over-delivered and an amount of insulin under-delivered during the basal rate test using the insulin delivery information and the sampled blood glucose data. The insulin calculation module 120 may recommend changes to the basal rate pattern. The controller 115 may communicate recommended changes to the basal rate pattern through the communication port 747 and/or the controller 115 may display the recommended changes.

### Method Embodiments

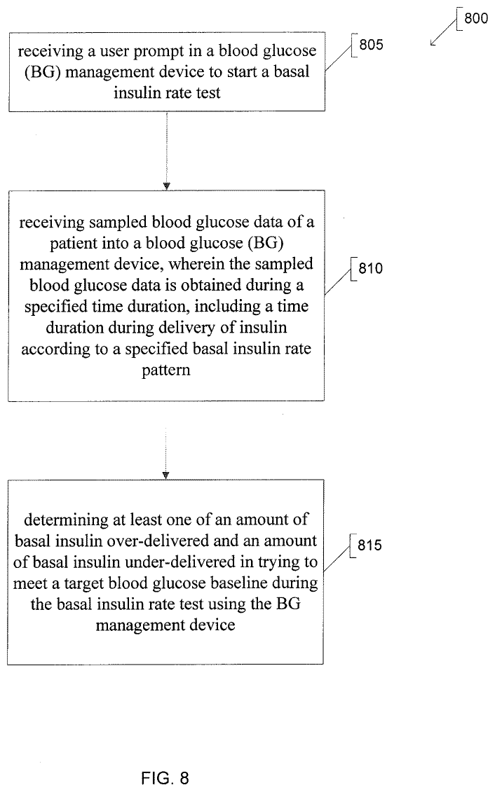

FIG. 8 is a flow diagram of a method 800 of using a BG management device to execute a basal rate test. At block 805, a user prompt is received in a BG management device to start a basal insulin rate test. The user interface may include a push-button, keypad, or mouse. The user interface may also include a display to display one or more instructions for the user to execute the basal rate test, and to display to display any recommend changes to a basal rate or a basal rate pattern. In some embodiments, the method 800 includes displaying instructions for the basal insulin rate test using the BG management device.

At block 810, sampled blood glucose data is received in the BG management device. The blood glucose data is obtained from a patient during a specified time duration, including a time during delivery of insulin according to a basal insulin rate pattern that is part of the basal rate test.

At block 815, at least one of an amount of basal insulin over-delivered or an amount of basal insulin under-delivered is determined. The over-delivery and/or under-delivery occur in trying to meet a target blood glucose baseline during the basal insulin rate test. In some embodiments, the method 800 includes the BG management device automatically recommending changes, if any, to the basal insulin rate pattern.

In some embodiments, the method 800 includes determining an amount of basal insulin over-delivered or an amount of basal insulin under-delivered using the correction factor and the variance from the blood glucose baseline concentration. In some embodiments, the amount of insulin over or under-delivered is determined using an adjusted correction factor. The correction factor may be adjusted using a correction factor multiplier. In some embodiments, recommending a change may include spreading out the change to the basal delivery rate pattern out over a time duration corresponding to a time to a change in direction of the blood glucose data values.

In some embodiments, the method includes recommending changes to the basal insulin rate pattern that precede any actual times of over-delivery or under-delivery by a time duration that compensates for a delay or lag time associated with subcutaneous insulin delivery. In some embodiments, the method 800 includes calculating the lag time using at least one of i) the type of insulin delivered during the basal rate test, ii) the activity level of the patient at the time the basal rate test takes place, iii) the fitness level of the patient, and iv) the method of obtaining the blood glucose data, e.g., whether the blood glucose data was obtained from blood or from interstitial fluid. In some embodiments, the method 800 includes calculating the lag time using blood glucose data obtained prior to the basal insulin rate test.

According to some embodiments, the BG management device includes an insulin pump. The method 800 includes determining an amount of active insulin (IOB) at the beginning of the basal rate test. The IOB may be determined before delivering basal insulin according to a basal rate pattern of the basal insulin test. In some embodiments, if an amount of active insulin is above a specified threshold active insulin amount, the BG management device may cancel the basal rate test. In some embodiments, the method 800 includes canceling the basal insulin rate test if an insulin bolus dose, such as a correction bolus or a carbohydrate bolus, is delivered during the basal insulin rate test.

According to some embodiments, the BG management device includes an insulin pump and a GM. The method 800 includes automatically receiving the sampled blood glucose data from the blood glucose monitor. In some embodiments, the BG management device includes the insulin pump and the blood glucose data is obtained using a separate device. The method 800 includes receiving the sampled blood glucose data into the BG management device from the separate device through a communication port. The communication port may be a wireless port or a wired port. The separate device may be a continuous GM.

In some embodiments, the separate device may be a GM that requires some action by the user to obtain a blood glucose reading. For example, the GM may require the user to place a test strip into the GM in order to obtain a glucose reading. In some embodiments, the method 800 may include prompting the user through a user interface to obtain blood glucose data using the separate device. The prompting may be periodic during the basal rate test.

In some embodiments, the blood glucose data obtained from the separate device is entered manually into the BG management device. The method 800 includes the BG management device receiving the blood glucose data through the user interface. The user interface is configured for manual entry of blood glucose data, such as by including a keypad and a display. The user reads the blood glucose data from the separate GM and manually enters the blood glucose data into the BG management device. In some embodiments, the method 800 includes the BG management device periodically prompting the user to manually enter a blood glucose value during the basal rate test.

According to some embodiments, the BG management device includes a GM and does not include an insulin pump. The basal insulin is delivered according to a basal rate pattern using a second separate device. The sampled blood glucose data is received automatically using the included GM. The method 800 further includes receiving information related to insulin delivery into the BG management device from the separate device, including an amount of insulin delivered according to the basal rate pattern. The BG management device determines at least one of an amount of insulin over-delivered and an amount of insulin under-delivered during the basal rate test using the insulin delivery information and the sampled blood glucose data.

In some embodiments, the method 800 includes receiving the insulin delivery information into the BG management device through a communication port. As part of the basal rate test, the BG management device may communicate a recommended change to the basal rate pattern to the separate device using the communication port. This is useful if the separate device is an insulin pump. In some embodiments, the method 800 includes receiving the insulin delivery information into the BG management device by manually entering the insulin delivery information. The information is manually entered via a user interface on the BG management device. Any recommended changes to the basal rate pattern may be displayed on the BG management device.

According to some embodiments, the BG management device does not include a GM or an insulin pump. The basal insulin is delivered according to a basal rate pattern using a second separate device, such as an insulin pump for example. The method 800 includes providing insulin delivery information, such as an amount of insulin delivered according to the basal rate pattern, to the BG management device using the second device.

The BG management device receives sampled blood glucose data from the second separate device or a third device. At least one of the insulin delivery information and the sampled blood glucose data includes a time-stamp to allow for alignment of the insulin delivery information and the blood glucose data. For example, the time-stamp for the insulin delivery may be the time at which the basal rate changes. The BG management device determines at least one of an amount of insulin over-delivered and an amount of insulin under-delivered during the basal rate test using the insulin delivery information and the sampled blood glucose data. Any recommended changes to the basal rate pattern may be displayed on the BG management device.

In some embodiments, the method 800 includes executing the basal insulin rate test during a substantially same time on multiple days. In some examples, the method 800 includes executing an overnight basal rate test. In some examples, the method includes executing an overnight basal rate test that includes an overnight basal rate calibration and verification technique.

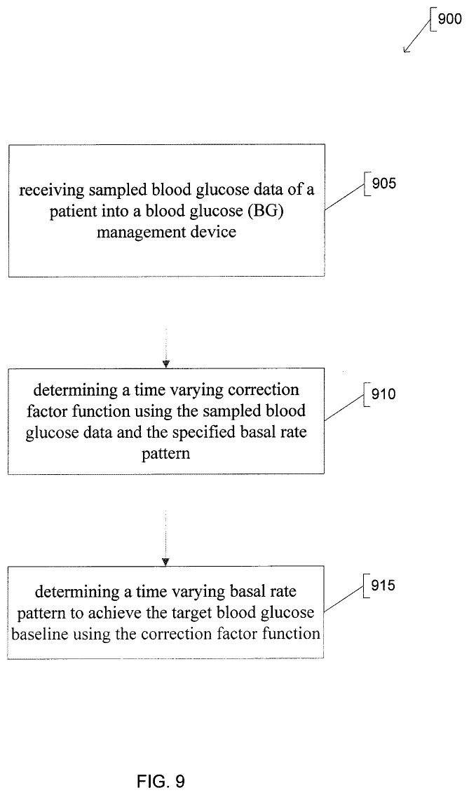

FIG. 9 is a flow diagram of another method 900 of using a BG management device to execute a basal rate test. At block 905, sampled blood glucose data is received in a BG management device. The blood glucose data may be obtained from a patient during a specified time duration according to a specified basal insulin rate pattern that is part of the basal rate test. At block 910, a time varying correction factor c(t) is determined using the sampled blood glucose data and the specified basal insulin rate pattern. At block 915, a time varying basal rate pattern b(t) is determined. The time varying basal rate pattern is to achieve the target blood glucose baseline. The target blood glucose baseline may be a constant or a time varying function g(t). In some embodiments, the method 900 includes generating a change to the test-specified basal rate pattern using the determined time varying basal rate pattern b(t). In some embodiments, the method includes recommending a change to the test-specified basal rate pattern, such as by using a display for example.

The accompanying drawings that form a part hereof, show by way of illustration, and not of limitation, specific embodiments in which the subject matter may be practiced. The embodiments illustrated are described in sufficient detail to enable those skilled in the art to practice the teachings disclosed herein. Other embodiments may be utilized and derived therefrom, such that structural and logical substitutions and changes may be made without departing from the scope of this disclosure. This Detailed Description, therefore, is not to be taken in a limiting sense, and the scope of various embodiments is defined only by the appended claims, along with the full range of equivalents to which such claims are entitled.

Such embodiments of the inventive subject matter may be referred to herein, individually and/or collectively, by the term “invention” merely for convenience and without intending to voluntarily limit the scope of this application to any single invention or inventive concept if more than one is in fact disclosed. Thus, although specific embodiments have been illustrated and described herein, it should be appreciated that any arrangement calculated to achieve the same purpose may be substituted for the specific embodiments shown. This disclosure is intended to cover any and all adaptations, or variations, or combinations of various embodiments. Combinations of the above embodiments, and other embodiments not specifically described herein, will be apparent to those of skill in the art upon reviewing the above description.

The Abstract of the Disclosure is provided to comply with 37 C.F.R. § 1.72(b), requiring an abstract that will allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own.

What is claimed is:

1. An infusion pump system, comprising:
a pump mechanism configured to facilitate delivery of insulin to a user;
a communications interface configured to receive glucose level data of the user from a glucose monitor; and
a processor functionally linked to the pump mechanism and the communications interface, the processor configured to:
cause the pump mechanism to deliver the insulin to the user based on the glucose level data received from the continuous glucose monitor;
determine that an additional amount of the insulin should be delivered to the user as a bolus;
automatically reduce the additional amount of the insulin in the bolus by a predetermined amount; and
deliver the bolus with the reduced additional amount of the insulin to the user as a bolus delivery of insulin.

2. The infusion pump system of claim 1, wherein the processor is configured to automatically reduce the additional amount of the insulin by the predetermined amount by reducing the additional amount of the insulin by a predetermined percentage.

3. The infusion pump system of claim 2, further comprising a memory and wherein the predetermined percentage is stored in the memory.

4. The infusion pump system of claim 1, wherein the at least one processor is configured to determine that the additional amount of the insulin should be delivered to the user based on the glucose level data.

5. The infusion pump system of claim 1, wherein the additional amount of the insulin is configured to maintain the glucose levels of the user at a target glucose level.

6. The infusion pump system of claim 5, wherein the target glucose level is a single glucose level.

7. The infusion pump system of claim 5, wherein the target glucose level is a range of glucose levels.

8. The infusion pump system of claim 1, wherein the processor is configured to automatically reduce the additional amount of the insulin by applying a multiplier to the additional amount of the insulin that should be delivered to the user.

9. The infusion pump system of claim 8, wherein the multiplier reduces the additional amount of the insulin by a predetermined percentage.

10. An infusion pump system, comprising:
a pump mechanism configured to facilitate delivery of insulin to a user;
a communications interface configured to receive glucose level data of the user from a glucose monitor; and
a processor functionally linked to the pump mechanism and the communications interface, the processor configured to:
cause the pump mechanism to deliver the insulin to the user based on the glucose level data received from the continuous glucose monitor;
determine that an additional amount of the insulin should be delivered to the user as a bolus;
automatically apply a multiplier to the additional amount of insulin to reduce the additional amount of insulin; and
deliver the bolus with the reduced additional amount of the insulin to the user as a bolus delivery of insulin.

11. The infusion pump system of claim 10, wherein the multiplier reduces the additional amount of insulin by a predetermined percentage.

12. The infusion pump system of claim 11, further comprising a memory and wherein the predetermined percentage is stored in the memory.

13. The infusion pump system of claim 10, wherein the at least one processor is configured to determine that the additional amount of the insulin should be delivered to the user based on the glucose level data.

14. The infusion pump system of claim 10, wherein the additional amount of the insulin is configured to maintain the glucose levels of the user at a target glucose level.

15. The infusion pump system of claim 14, wherein the target glucose level is a single glucose level.

16. The infusion pump system of claim 14, wherein the target glucose level is a range of glucose levels.

17. An infusion pump system, comprising:
a pump mechanism configured to facilitate delivery of insulin to a user;
a communications interface configured to receive glucose level data of the user from a glucose monitor; and
a processor functionally linked to the pump mechanism and the communications interface, the processor configured to:
cause the pump mechanism to deliver the insulin to the user based on the glucose level data received from the continuous glucose monitor;
determine that an additional amount of the insulin should be delivered to the user as a bolus; and
deliver a bolus to the user that is a predetermined percentage of the additional amount of the insulin.

18. The infusion pump system of claim 17, wherein the additional amount of the insulin is configured to maintain the glucose levels of the user at a target glucose level.

19. The infusion pump system of claim 18, wherein the target glucose level is a single glucose level.

20. The infusion pump system of claim 18, wherein the target glucose level is a range of glucose levels.
